(19) United States
(12) Patent Application Publication
(10) Pub. No.: US 2008/0207992 A1
(43) Pub. Date: Aug. 28, 2008

(54) Microsurgical Illuminator with Adjustable Illumination

(75) Inventors: Gregg D. Scheller, Glencoe, MO (US); Timothy J. Nadolski, Webster Groves, MO (US); Andre Maia, Osasco (BR); Carl Awh, Nashville, TN (US)

Correspondence Address: THOMPSON COBURN, LLP, ONE US BANK PLAZA, SUITE 3500, ST LOUIS, MO, 63101, US

(73) Assignee: SYNERGETICS, INC., St. Charles, MO (US)

(21) Appl. No.: 11/680,321

(22) Filed: Feb. 28, 2007

(51) Int. Cl. A61B 1/00

(52) U.S. Cl. 600/101

(57) ABSTRACT

An adjustable ophthalmic surgery chandelier illuminator has a glass optic fiber with a conical exterior surface at its distal end that disburses illumination in the interior of the eye. The glass fiber is contained in a retractable needle that has a long, sharp beveled surface that facilitates insertion of the needle and the optic fiber into the eye, and then is retracted relative to the fiber distal end to adjust the field of illumination inside the eye.

### BACKGROUND OF THE INVENTION

1. Field of the Invention

The present invention pertains to an ophthalmic surgery apparatus that provides wide field illumination to the interior of the eye, where the degree of illumination is adjustable. More specifically, the present invention provides an ophthalmic surgery chandelier illuminator that is comprised of a glass optic fiber with a conical surface that disburses illumination in the interior of the eye, and a retractable needle mounted over the fiber conical surface. The needle has a long, sharp beveled surface that facilitates insertion of the needle and optic fiber into the eye, and can then be retracted relative to the fiber conical surface to adjust the field of illumination inside the eye.

2. Description of the Related Art

In the practice of ophthalmic surgery, a chandelier illuminator is a microsurgical instrument that is used to provide a wide field of illumination in the interior of the eye. Chandeliers of the prior art typically comprise an optic fiber having an elongate length between opposite proximal and distal ends. The optic fiber is typically a plastic (PMMA) fiber. The proximal end of the fiber is provided with a connector that connects the fiber to a separate light source for transmitting illumination light through the fiber length. The distal end of the optic fiber is typically given a shape that provides wide-field illumination, usually a cone shape. The instrument is also provided with some means of introducing the distal end of the fiber inside the eye, for example by inserting the instrument distal end through an incision in the top of the eye, or inserting the instrument distal end through a cannula that is positioned in the eye for a surgical procedure.

A number of problems have been experienced in the use of prior art chandelier illuminators. For example, when making an incision in the eye with a sharp trocar for later insertion of the chandelier illuminator through the incision, it is necessary to displace the conjunctiva to position the incision at the top of the eye. The conjunctiva is a mucus membrane that lines the inner surface of the eyelid and the exposed surface of the eyeball beneath the eyelid. In inserting a chandelier illuminator in this manner, it is necessary for the surgeon to hold the conjunctiva in its displaced position while making the incision in the eye, and then later inserting the chandelier illuminator through the incision. If the conjunctiva is not held in its displaced position, the membrane will spring back over the eye covering the incision and making it difficult for the surgeon to find the location of the incision to insert the chandelier illuminator.

As a further example of difficulties associated with using prior art chandelier illuminators, the microsurgical instruments typically used in ophthalmic surgery and in particular a vitrectomy are typically packaged in a sterilized pack. The typical sterilized pack contains only three cannulas that are inserted into the eye. The cannulas provided are generally used for an illumination device, a surgical laser device or a gripping device, and a source of infusion. This does not leave any open cannulas to be used for insertion of the chandelier illuminator into the interior of the eye.

A still further disadvantage experienced with prior art chandelier illuminators is that most of the current chandeliers are made of a plastic (PMMA) optic fiber. In recent years, the intensity of the light supplied by the separate illumination light source to which the chandelier is connected has increased. The increase in the intensity of the illumination light has become problematic in that the distal tip of the plastic optic fiber that disburses the light in the eye interior has the possibility of melting, which could cause damage to the eye wall.

Lastly, a further disadvantage of prior art chandelier illuminators has been experienced during a fluid/air exchange of a vitrectomy. During the fluid/air exchange, the difference in the refractive indices between the fluid and the air causes the prior art chandelier illuminator to produce glare in the eye interior, making it difficult for the surgeon to visualize the internal structures of the eye.

### SUMMARY OF THE INVENTION

The adjustable ophthalmic surgery chandelier illuminator of the present invention addresses all of the disadvantages associated with prior art chandelier illuminators set forth above. The chandelier illuminator of the invention is basically comprised of a shaped glass optic fiber that is contained inside a retractable needle.

The glass optic fiber has an elongate, flexible length with opposite proximal and distal ends. A light source connector is provided at the fiber proximal end and is adapted to removably attach the fiber proximal end to an illumination light source for transmission of illumination light through the length of the glass fiber. The distal end of the optic fiber is provided with an exterior surface configuration that disburses the light transmitted through the fiber. In the preferred embodiment, the shaped distal end surface of the optic fiber has a cone configuration. The exterior surface of the optic fiber between the light source connector and the shaped distal end surface is surrounded by polyimide tubing. Should the glass fiber fracture during bending movements, the polyimide tubing securely holds together the two adjacent pieces of the glass fiber on opposite sides of the fracture.

A straight tubular needle is mounted on the optic fiber for sliding movement. The needle is positioned adjacent the optic fiber distal end. A first end of the needle is positioned toward the optic fiber proximal end, and a second end of the needle is positioned adjacent the shaped distal end surface of the optic fiber. The second end of the needle is provided with a sharp beveled surface.

A needle housing is secured to the optic fiber adjacent the optic fiber distal end. The housing has a hollow interior bore and window openings in opposite sides of the housing. The optic fiber and the needle extend through the housing bore, with the needle first end being positioned in the housing bore. The window openings are positioned on opposite sides of the needle first end.

A slide bar is secured to the needle adjacent the needle first end. Opposite ends of the slide bar extend from the needle first end through the pair of window openings in the housing. These opposite ends of the slide bar are positioned outside of the housing where they are accessible by the surgeon for gripping and manipulating the slide bar through the housing interior bore. Moving the slide bar forward through the housing toward the optic fiber distal end causes the needle to move over the optic fiber distal end, containing the conical exterior surface of the optic fiber in the interior of the needle. Moving the slide bar rearward or toward the optic fiber proximal end causes the needle to be retracted over the optic fiber distal end surface, exposing the fiber distal end surface from the beveled surface of the needle.

During use of the adjustable chandelier illuminator, the slide bar is moved forward positioning the long, sharp beveled end surface of the needle over the optic fiber conical surface. The long beveled surface of the needle is required for ease of insertion of the optic fiber conical end surface into the eye. With the beveled surface of the needle extending past the conical surface of the fiber and the conical surface positioned in the needle, the needle beveled end surface is inserted fully into the eye at the desired position. With the needle so inserted, the slide bar can be manually manipulated rearwardly to retract the needle to a desired extent, adjustably exposing the shaped exterior surface of the optic fiber distal end. The ability to retract the needle relative to the optic fiber distal end rather than extending the optic fiber distal end from the needle minimizes the length of the instrument positioned inside the eye. A longer extension of the instrument inside the eye would present an increased chance that the eye lens could be damaged, causing a cataract. The shaped exterior surface of the glass fiber is immune to melting due to the intensity of the illumination light, and the needle position can be manually adjusted to provide the surgeon with a desired amount of shielding of the illumination light disbursed by the optic fiber distal end. During a fluid/air exchange, the needle can be extended until the tip of the optic fiber is no longer visible to the surgeon, reducing the glare in the interior of the eye while providing adequate illumination.

The ophthalmic surgery adjustable chandelier illuminator provides a sharp needle trocar and an optic fiber chandelier incorporated into a single microsurgical instrument. This enables the surgeon to position a wide-field chandelier illuminator in a patient's eye with fewer steps, and thereby facilitates the use of the instrument. The use of a glass optic fiber eliminates the potential danger of the fiber melting due to intense illumination light. The retractable needle and the long beveled surface of the needle enables the easy insertion of the instrument into the eye and provides for adjustable shielding of the illumination provided by the instrument. The long beveled surface of the needle enables the positioning of the shaped exterior surface of the optic fiber inside the needle during needle insertion, and provides the illumination shield on the needle that can be adjustably positioned relatively to the optic fiber distal end surface to adjust the field of illumination provided by the optic fiber distal end surface.

### BRIEF DESCRIPTION OF THE DRAWINGS

Further features of the invention are set forth in the following detailed description of the preferred embodiment of the invention and in the drawing figures.

FIG. 1 is a plan view of the adjustable ophthalmic surgery chandelier illuminator of the invention.

FIG. 2 is a partial, enlarged cross-section view of the distal end of the illuminator.

FIG. 3 is a view similar to that of FIG. 2, but with the instrument rotated 90 degrees.

### DETAILED DESCRIPTION OF THE PREFERRED EMBODIMENT

The adjustable ophthalmic surgery chandelier illuminator of the invention is basically comprised of an optic fiber 10, a needle 12, a needle housing 14, and means 16, 18 for holding the illuminator in a desired position in use. Unless set forth otherwise herein, the materials used for constructing each of these component parts of the illuminator are those typically used in the construction of prior art ophthalmic surgery illuminators.

In the preferred embodiment, the optic fiber 10 is a glass or silica optic fiber. The fiber 10 has an elongate, flexible, continuous length that extends between a proximal end 20 of the fiber and distal end 22 of the fiber. The optic fiber 10 is preferably a 25 gauge optic fiber, but could also be a 20, 23, and 27 gauge fiber. The length of the optic fiber 10 is sufficiently long to enable the illuminator of the invention to be easily manipulated by a surgeon.

A light source connector is provided on the optic fiber proximal end 20. The connector is comprised of a length of metal tubing 24 and a cylindrical plastic handle 26. The construction of the light source connector shown in the drawings is only one example of a light source connector that could be used with the adjustable chandelier illuminator of the invention, and should not be interpreted as limiting. Depending on the illumination light source with which the illuminator is used, the construction of the connector would change to adapt the illuminator for use with the particular light source. As is typical in the construction of light source connectors, the optic fiber proximal end 20 is positioned at the end of the connector tubing 24 to properly position the optic fiber proximal end relative to the light source when the connector is connected to the light source.

The opposite distal end 22 of the optic fiber is provided with a distal end portion 28 having a shaped exterior surface 32. The exterior surface 32 can be described generally as being a converging surface and as having a cross-sectional area that decreases as the fiber distal end portion extends to the fiber distal end 22. In the preferred embodiment, the exterior surface 32 of the fiber distal end portion 28 is conical. However, the fiber distal end portion 28 could have a bullet shape, and could be provided with a flat beveled surface on one side of the fiber. In addition, the fiber distal end could have a separate light diffusing optic attached to the end.

The remaining exterior surface of the optic fiber 10 extending from the distal end portion exterior surface 32 to the light source connector 24, 26 is engaged by a length of polyimide tubing 34. The polyimide tubing 34 surrounds and securely engages around the optic fiber 10. The particular polyimide tubing 34 is employed to securely engage with the exterior surface of the optic fiber 10 so that, should the glass fiber become fractured at some point along the length of the fiber, the polyimide tubing securely holds together the two portions of the optic fiber 10 on the opposite sides of the fracture, thereby allowing illumination light to be transmitted through the optic fiber 10 and through the fracture in the fiber.

The tubular needle 12 is mounted on the optic fiber 10 adjacent to the fiber distal end 22 for sliding movement of the needle over the fiber. As shown in FIG. 2, the needle 12 is actually mounted on a portion of the polyimide tubing 34. The needle 12 has a center axis 36 that is coaxial with a center axis of the portion of the optic fiber 10 contained in the needle. The needle 12 extends from a first end surface 38 of the needle positioned toward the optic fiber proximal end 20, to a second end surface 40 of the needle positioned toward the optic fiber distal end 22. The first end surface is a flat circular surface that is perpendicular to the needle center axis 36. The needle second end surface 40 is a sharp beveled surface that is positioned in a plane oriented at an oblique angle relative to the center axis 36. An upper portion of the second end surface 40 as shown in FIG. 2 functions as a shield 42 that extends over the optic fiber conical surface 32. The sharp bevel of the needle second end surface 40 extends or projects the shielding portion 42 of the surface axially beyond the portion of the needle surface 44 at the diametrically opposite side of the axis 36. In the preferred embodiment of the invention the needle 12 is constructed of surgical stainless steel, for example, the same type of steel employed in the construction hypodermic needles.

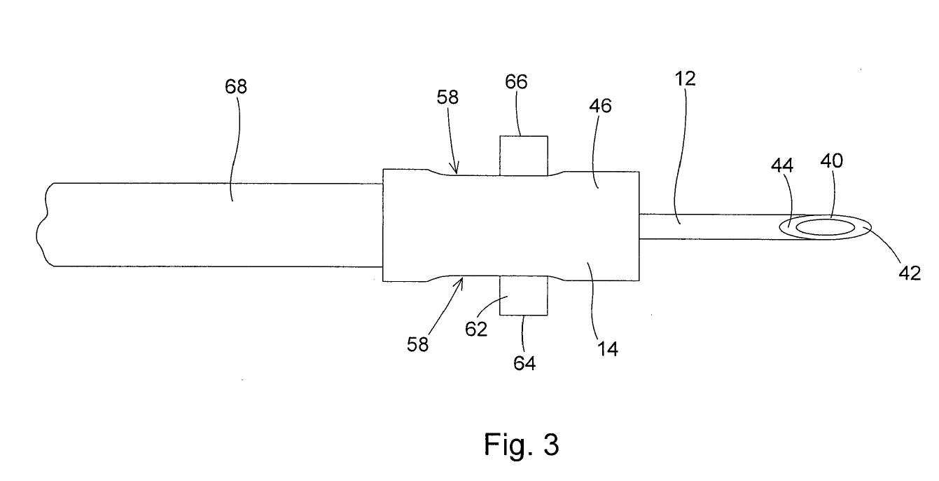

The needle housing 14 has a cyclical configuration with a generally cylindrical exterior surface 46. A smaller cylindrical neck 48 projects from one end of the housing 14. A narrow interior bore 50 extends through the housing 14. The bore 50 intersects with a larger, hollow interior bore cavity 52 inside the housing. The optic fiber 10 extends through the housing bore 50 and through the housing cavity 52. The needle 12 extends from the needle first end 38 positioned in the housing cavity 52, through a portion of the housing interior bore 50 and out of the housing. A guide, for example an o-ring guide 54 engages between the housing interior bore 50 and the exterior of the needle 12. A ring 56 secures the guide 54 to the housing 14. The needle 12 is free to slide through the housing 14 and over the optic fiber 10. The needle 12 is movable between a first, extended position of the needle 12 shown in FIG. 3, and a second, retracted position of the needle 12 shown in FIG. 2. The housing 14 also has a pair of oblong window openings 58 through opposite sides of the housing. The openings 58 open into the interior cavity 52 of the housing.

A slide bar 62 is mounted on the needle 12. The slide bar 62 is received in the housing cavity 52 and window openings 58 for sliding movement through the cavity and windows. The slide bar 62 is secured to the needle 12 adjacent the needle first end 38. The slide bar 62 has opposite ends 64, 66 that project from the housing cavity 52, through the housing window openings 58, 60 to the exterior of the housing. The slide bar ends 64, 66 are accessible outside of the housing 14 for gripping by the surgeon to move the slide bar 62 relative to the housing 14. Movement of the slide bar 62 forwardly through the housing cavity 52 and the window openings 58 causes the needle 12 to be moved to its first, extended position, and movement of the slide bar rearwardly in the housing 14 causes the needle 12 to be moved to its second, retracted position.

An exterior, protective length of tubing 68 extends between the needle housing 14 and the light source connector handle 26. The tubing 68 has a distal end 70 that is secured over the housing neck 48. The opposite proximal end of the tubing (not shown) extends into and is secured inside the light source connector handle 26.

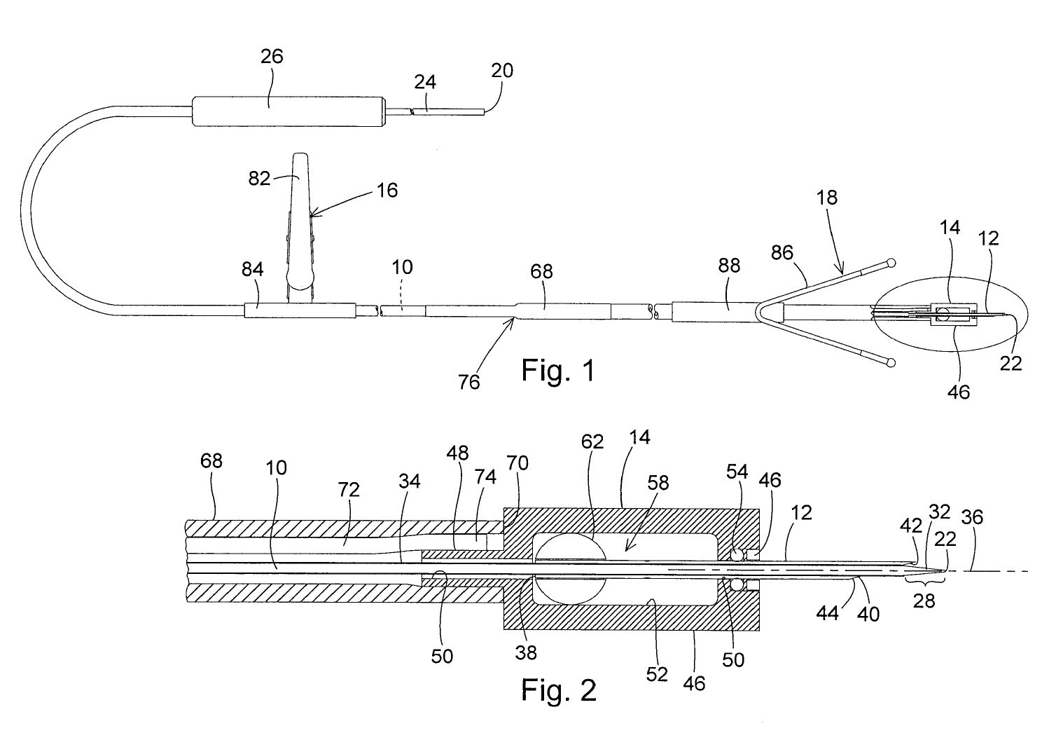

A length of wire 72 is secured inside the exterior tubing 68. The wire 72 has a distal end 74 that is secured to the needle housing 14 by the tubing distal end 70. The wire 72 extends along a portion of the length of the exterior tubing 68 to a proximal end of the wire (not shown) that is positioned at the reduction in the diameter 76 of the exterior tubing shown in FIG. 1. The wire 72 is malleable, and is used to bend the portion of the tubing 68 containing the wire to position the illuminator in a desired position.

The illuminator is also provided with additional means of holding the illuminator in a desired position. The additional means includes a manually operated clip 82 mounted on a sleeve 84 that is adjustably positionable along the length of the exterior tubing 68. The means also include a V-shaped stabilizer or bipod support 86 that is mounted on a sleeve 90 that is adjustably positionable along the length of the exterior tubing 68.

As stated earlier, the illuminator is used in providing a wide-field of illumination in the interior of the eye during an ophthalmic surgery procedure. In use of the instrument, the slide bar 62 is first manipulated to position the needle 12 forward relative to the housing 14 and the optic fiber distal end 22. This positions the conical optic fiber distal end surface 32 in the interior of the needle 12. The needle 12 is then positioned in the eye by inserting the beveled end surface 40 through the eye, without requiring a prior incision or a prior insertion of a cannula. With the needle 12 fully inserted into the eye, the slide bar 62 can be manually manipulated by the surgeon to gradually retract the needle 12 over the optic fiber distal end portion 28. This gradually exposes the conical exterior surface 32 of the optic fiber distal end portion 28 from the interior of the needle 12, and gradually adjusts the illumination transmitted to the interior of the eye. If needed, the needle beveled end surface 40 can be employed as a shielding surface to shield the view of the surgeon from the illumination light transmitted from the optic fiber distal end surface 32. The V-shaped support 86 and the clip 82, as well as, the malleable wire 72 may be used to secure the illuminator in a desired position relative to the eye after insertion of the illuminator in the eye.

Although the adjustable ophthalmic surgery chandelier illuminator of the invention has been described above by reference to a particular embodiment of the invention, it should be understood that modifications and variations could be made to the illuminator without departing from the intended scope of the following claims.

What is claimed is:

1. A microsurgical illuminator apparatus comprising:
an optic fiber having a flexible, elongate length with opposite proximal and distal ends;
a light source connector at the optic fiber proximal end, the light source connector being configured to be connected to a separate light source to enable illumination light to be transmitted through the length of the optic fiber to the optic fiber distal end;
a means for dispersing illumination light at the optic fiber distal end, the means dispersing illumination light transmitted through the optic fiber from the optic fiber distal end; and,
a tubular needle mounted on the optic fiber for movement of the needle over the optic fiber, the needle having a length with opposite first and second ends with the needle first end being positioned on the optic fiber toward the optic fiber proximal end and the needle second end being positioned on the optic fiber toward the optic fiber distal end, the needle having a beveled end surface at the second end.

2. The apparatus of claim 1, further comprising:
the means for dispersing illumination light being a converging exterior surface on the optic fiber at the optic fiber distal end.

3. The apparatus of claim 2, further comprising:
the needle being movable over the optic fiber between first and second positions of the needle relative to the optic fiber where in the first position the converging exterior surface of the optic fiber is contained inside the needle between the needle first and second ends and in the second position the converging exterior surface of the optic fiber is outside of the needle and projects from the needle second end.

4. The apparatus of claim 3, further comprising:
the needle having a center axis and the needle beveled end surface being positioned in a place that is oriented at an oblique angle relative to the needle center axis.

5. The apparatus of claim 4, further comprising:
the optic fiber converging exterior surface being symmetrical around the needle center axis.

6. The apparatus of claim 4, further comprising:
the needle length being straight; and,
the optic fiber projecting straight from the needle second end when the needle is in the second position of the needle relative to the optic fiber.

7. The apparatus of claim 2, further comprising:
the optic fiber being no larger than a 20 gauge optic fiber.

8. The apparatus of claim 2, further comprising:
the optic fiber being a silica fiber; and,
a length of polyimide tubing engaging around an exterior surface of the optic fiber and extending from the light source connector to the converging exterior surface.

9. The apparatus of claim 2, further comprising:
a housing mounted on the optic fiber and the tubular needle, the optic fiber being stationary relative to the housing and the needle being movable relative to the housing between the first and second positions of the needle, the housing having a pair of window openings in opposite sides of the housing; and
a slide bar mounted in the housing for movement of the slide bar relative to the housing, the slide bar having opposite first and second ends that project through the pair of window openings in the opposite sides of the housing to an exterior of the housing where the slide bar first and second ends can be manually gripped and moved, and the slide bar being secured to the needle to cause the needle to move between the first and second positions of the needle in the response to the slide bar being manually moved.

10. A microsurgical illuminator apparatus comprising:
an optic fiber having a flexible, elongate length with opposite proximal and distal ends, a distal end portion of the optic fiber adjacent the optic fiber distal end having a cross sectional area that reduces as the distal end portion of the optic fiber extends to the optic fiber distal end forming an exterior surface on the distal end portion that is shaped to disperse illumination light transmitted through the optic fiber;
a light source connector at the optic fiber proximal end, the light source connector being configured to be connected to a separate light source to enable illumination light to be transmitted through the length of the optic fiber to the optic fiber distal end portion and dispersed from the exterior surface of the optic fiber distal end portion; and,
a needle mounted on the optic fiber for movement of the needle over the optic fiber, the needle having a tubular length with opposite first and second ends with the needle first end being positioned on the optic fiber toward the optic fiber proximal end and the needle second end being positioned on the optic fiber toward the optic fiber distal end, the needle having a beveled end surface at the needle second end, the needle being movable over the optic fiber between first and second positions of the needle relative to the optic fiber where in the first position the optic fiber distal end portion is contained inside the needle between the needle first and second ends and in the second position of the needle the optic fiber distal end portion is outside the needle and projects from the needle second end.

11. The apparatus of claim 10, further comprising:
the needle length being straight and having a center axis and the needle beveled end surface being positioned in a plane that is oriented at an oblique angle relative to the needle center axis.

12. The apparatus of claim 11, further comprising:
the optic fiber distal end portion having a center axis that is coaxial with the needle center axis, and the optic fiber distal end portion being symmetric about the optic fiber distal end portion center axis.

13. The apparatus of claim 11, further comprising:
the optic fiber distal end portion projecting straight along the needle center axis when the needle is moved to the second position.

14. The apparatus of claim 10, further comprising:
the optic fiber being no larger than a 20 gauge optic fiber.

15. The apparatus of claim 10, further comprising:
the optic fiber being a silica optic fiber and having a length of polyimide tube engaging around an exterior surface of the optic fiber and extending from the light source connector to the distal end portion of the optic fiber.

16. The apparatus of claim 10, further comprising:
a housing having a hollow interior bore and an exterior surface, the housing being mounted on the optic fiber and the needle with the optic fiber being stationary in the housing interior bore and the needle being movable in the housing interior bore, the housing having a pair of window openings in the housing exterior surface on opposite sides of the housing and on opposite sides of the optic fiber and the needle; and
a slide bar mounted in the housing interior bore for movement of the slide bar through the housing interior bore, the slide bar being secured to the needle in the housing interior bore and the slide bar having opposite ends the project from the housing interior bore through the pair of window openings to outside the housing where the slide bar opposite ends are accessible for manual gripping and moving the slide bar to move the needle between the first and second positions of the needle.

17. The apparatus of claim 10, further comprising:
means on the optic fiber for holding the distal end portion of the optic fiber stationary relative to an eye.

18. The apparatus of claim 10, further comprising:
the optic fiber distal end portion having a conical exterior surface shape.

19. A microsurgical illuminator apparatus comprising:
an optic fiber having a flexible elongate length with opposite proximal and distal ends, the optic fiber having a converging exterior surface at the optic fiber distal end, the converging exterior surface being shape to disperse illumination light transmitted through the length of the optic fiber;
a light source connector at the optic fiber proximal end, the light source connector being configured to be connected to a separate light source to enable illumination light to be transmitted through the length of the optic fiber to the converging exterior surface of the optic fiber where the illumination light is dispersed from the converging exterior surface; and,
a needle mounted on the optic fiber for movement of the needle over the optic fiber, the needle having a tubular length with a center axis and axially opposite first and second ends, the needle first end being positioned on the optic fiber toward the optic fiber proximal end and the needle second end being positioned on the optic fiber toward the optic fiber distal end, the needle second end having an end surface with a shield portion on one side of the needle center axis that extends axially past a portion of the end surface on an opposite side of the needle center axis, the needle being movable over the optic fiber between first and second positions of the needle relative to the optic fiber where in the first position the optic fiber converging exterior surface is contained inside the needle between the needle first and second ends and in the second position the optic fiber converging exterior surface is outside the needle.

20. The apparatus of claim 19, further comprising:
the optic fiber converging exterior surface being a conical surface.

21. The apparatus of claim 19, further comprising:
the optic fiber being no larger than a 20 gauge optic fiber.

22. The apparatus of claim 19, further comprising:
the optic fiber being a silica optic fiber and the optic fiber having a length of polyimide tube engaging around an exterior surface of the optic fiber and extending along the length of the optic fiber from the light source connector to the converging exterior surface of the optic fiber.

23. The apparatus of claim 19, further comprising:
a housing having a hollow interior bore and an exterior surface, the housing being mounted on the optic fiber and the needle with the optic fiber being stationary in the housing interior bore and the needle being movable in the housing interior bore, the housing having a pair of window openings in the housing exterior surface on opposite sides of the housing and on opposite sides of the optic fiber and the needle; and
a slide bar mounted in the housing interior bore for movement of the slide bar through the housing interior bore, the slide bar being secured to the needle in the housing interior bore and the slide bar having opposite ends the project from the housing interior bore through the pair of window openings to outside the housing where the slide bar opposite ends are accessible for manual gripping and moving the slide bar to move the needle between the first and second positions of the needle.

24. The apparatus of claim 19, further comprising:
means on the optic fiber for holding the distal end portion of the optic fiber stationary relative to an eye.

25. A method of providing wide-field illumination in an interior of an eye in an ophthalmic surgery procedure, the method comprising:
providing an optic fiber with an elongate flexible length between opposite proximal and distal ends of the optic fiber;
providing a light service connector at the optic fiber proximal end;
providing a converging exterior surface at the optic fiber distal end;
positioning a tubular needle with a beveled end surface on the optic fiber for sliding movement of the needle between first and second positions of the needle relative to the optic fiber where in the first position the optic fiber converging exterior surface is positioned inside the needle and in the second position the optic fiber converging exterior surface is positioned outside the needle and extending from the needle beveled end surface;
positioning the needle in the first position;
connecting the light source connector to the source of illumination light to transmit illumination light through the optic fiber to the converging exterior surface at optic fiber distal end;
inserting the needle beveled end surface into an eye and positioning the optic fiber converging exterior surface inside the needle inside the eye;
securing the optic fiber stationary relative to the eye; and,
moving the needle from the first position to the second position and extending the optic fiber converging exterior surface from the needle beveled end surface and dispersing illumination light from the optic fiber converging exterior surface inside the eye.

26. The method of claim 25, further comprising:
providing the optic fiber as a silia glass fiber along the length of the optic fiber; and,
providing the optic fiber with a polyimide tubing on an exterior surface of the optic fiber extending from the light source connector to the converging exterior surface.

27. The method of claim 25, further comprising:
adjustably positioning the needle between the first and second positions and thereby adjusting an intensity of light dispersed inside the eye.

28. The method of claim 25, further comprising:
adjusting a position of the needle beveled end surface relative to the optic fiber converging exterior surface to adjust an intensity of illumination inside the eye.

29. The method of claim 25, further comprising:
providing a slide bar secured to the needle first end and manually gripping and moving the slide bar relative to the optic fiber to move the needle from the first position to the second position.
